(19) United States
(12) Patent Application Publication
(10) Pub. No.: US 2025/0312619 A1
(43) Pub. Date: Oct. 9, 2025

(54) Source dispensing unit for a needle assemblage

(71) Applicant: Alpha Tau Medical Ltd., Jerusalem (IL)

(72) Inventors: Sraya Makovsky, Beit Shemesh (IL); Shahar Polak, Meitar (IL); Niv Dana, Hod HaSharon (IL); Ofer Magen, Hod Hasharon (IL)

(21) Appl. No.: 19/032,503

(22) Filed: Jan. 21, 2025

Related U.S. Application Data
(60) Provisional application No. 63/631,502, filed on Apr. 9, 2024.

(51) Int. Cl. A61B 5/15; A61B 5/151

(52) U.S. Cl. CPC A61N 5/1007; A61N 2005/1008

(57) ABSTRACT

Apparatus for control of a needle assemblage having a handle coupled to a hollow needle and a stylet penetrating the hollow needle. The apparatus includes a retaining bar and a connector slidingly mounted on the retaining bar that is configured to fixedly attach to the handle. The connector has a control which in a first position causes translation of the connector proximally on the retaining bar by a preset distance to translate the handle and the hollow needle proximally by the preset distance and to translate the stylet distally by the preset distance with respect to the connector. In a second position the control causes translation of the connector on the retaining bar by the preset distance to translate the handle, the hollow needle, and the stylet by the preset distance.

### CROSS-REFERENCE TO RELATED APPLICATION

This application claims the benefit of U.S. Provisional Patent Application 63/631,502, filed Apr. 9, 2024, which is incorporated herein by reference.

### FIELD OF THE INVENTION

The present invention relates generally to endoscopy, and specifically to apparatus to implement the endoscopic delivery of a plurality of implants or substances.

### BACKGROUND OF THE INVENTION

In an endoscopic procedure, sources, for example implants or substances, may be placed in a patient for a variety of potential therapeutic uses, such as for the placement of fiducial markers, for drug delivery, and for the implantation of radiotherapy seeds. By way of an example, where endoscopy is used in a radiotherapy setting such as the Diffusing Alpha-emitters Radiation Therapy (DaRT) process, such sources are specifically formed as radioactive seeds which are implanted via an endoscope into a tumor in order to kill the tumor.

The correct positioning of the sources in the patient is extremely important, since even small deviations from a targeted position may not only significantly decrease the effectiveness of the procedure, but may also lead to deleterious side-effects such as damaging healthy tissue.

U.S. Pat. No. 10,292,786 to Clancy et al., describes a fiducial deployment system with a handle configured for actuation of the system. A fiducial may include one or more protuberances configured to engage one or more slots in a needle of the system. The needle may be configured to deliver a plurality of fiducials to a target location in serial fashion, one at a time.

U.S. Patent Application 2011/190662, to McWeeney, describes a device for needle biopsy and delivery of a diagnostic or therapeutic agent. The device includes a handle member having proximal and distal portions. A proximal handle member is disposed to the proximal portion of the handle member and a distal handle member is disposed to the distal portion of the handle member. A sheath lumen is disposed within the handle member.

U.S. Patent Application 2015/0031935, to Wazer et al., describes an instrument used for brachytherapy delivery in the treatment of cancer by radiation therapy. The instrument includes a handle having first and second handle actuators; an end effector, and an instrument shaft that connects the handle with the end effector. The end effector has first and second adjacent disposed Staple cartridges that each retain a set of Staples.

U.S. Pat. No. 10,589,071 to Subramanian, describes a dual double balloon catheter that includes a catheter having a proximal end portion, a central portion and a distal end portion. The catheter includes a plurality of lumens within the catheter extending from the proximal end portion, and a plurality of inflatable balloons positioned in the central portion and/or the distal end portion.

### SUMMARY OF THE INVENTION

An embodiment of the present invention provides an apparatus for control of a needle assemblage having a handle coupled to a hollow needle and a stylet penetrating the hollow needle, the apparatus including:
a retaining bar;
a connector slidingly mounted on the retaining bar, configured to fixedly attach to the handle, the connector having a control which:
in a first position causes translation of the connector proximally on the retaining bar by a preset distance to translate the handle and the hollow needle proximally by the preset distance and to translate the stylet distally by the preset distance with respect to the connector, and
in a second position causes translation of the connector on the retaining bar by the preset distance to translate the handle, the hollow needle, and the stylet by the preset distance.

The assemblage may have a static rod coupled to a sheath surrounding the hollow needle, the apparatus further including a further connector, slidingly mounted on the retaining bar, configured to fixedly connect to the static rod.

In a disclosed embodiment the connector includes a rack and a pinion, the pinion being coupled to a gear train retaining the stylet within the hollow needle. In the first position the rack and the pinion may engage, and in the second position the rack and the pinion may disengage.

In a further disclosed embodiment the apparatus incudes a rack enclosure wherein a rack is configured to slide, and wherein the control is configured to rotate the rack enclosure to a first location that places the rack in the first position and to a second location that places the rack in the second position. The rack enclosure may rotate about a hinge fixed to the retaining bar.

There is further provided, according to an embodiment of the present invention, a method for implanting at least one source into a patient, the method including:
providing a hollow needle having a needle distal end and a lumen configured to receive the at least one source;
inserting a stylet into the lumen;
inserting the at least one source into the lumen;
inserting the hollow needle into the patient so that the needle distal end is at a predetermined location within the patient;
feeding the stylet through the lumen to contact a proximal end of the at least one source and thereby push the at least one source so that a distal end of the at least one source aligns with the needle distal end; and
subsequent to feeding the stylet through the lumen to contact the proximal end of the at least one source, retracting the hollow needle proximally, while maintaining the stylet in position, so that the at least one source enters the patient at the predetermined location.

There is further provided, according to an embodiment of the present invention, a method for controlling a needle assemblage having a handle coupled to a hollow needle and a stylet penetrating the hollow needle, the method including:
slidingly mounting a connector on a retaining bar;
fixedly attaching the connector to the handle; and
attaching a control, having a first position and a second position, to the connector, wherein the control:
in the first position causes translation of the connector proximally on the retaining bar by a preset distance to translate the handle and the hollow needle proximally by the preset distance and to translate the stylet distally by the preset distance with respect to the connector, and
in the second position causes translation of the connector on the retaining bar by a given distance to translate the handle, the hollow needle, and the stylet by the given distance.

The present disclosure will be more fully understood from the following detailed description of the embodiments thereof, taken together with the drawings, in which:

### BRIEF DESCRIPTION OF THE DRAWINGS

FIGS. 1A and 1B are schematic illustrations of a needle assemblage, and of a source dispensing unit that may be attached to the needle assemblage, according to an embodiment of the present invention;

FIG. 2 is a schematic illustration of elements that are used by the needle assemblage, according to an embodiment of the present invention; and

FIGS. 3A-3C are different illustrations of a handle connector mounted on a bar, according to an embodiment of the present invention.

### DETAILED DESCRIPTION OF EMBODIMENTS

### Overview

In an endoscopic procedure to kill a tumor by implantation of sources comprising radioactive seeds, it is important to implant the seeds within the tumor as precisely as possible so as to prevent damage to tissue other than the tumor. While a physician may manually use a Fine Needle Aspiration (FNA)/Biopsy needle with a stylet to perform the implantation, the physician typically needs to have a high level of expertise for successfully implanting seeds, and even then the requirements of accurate positioning of the seeds, typically within 4 mm, are hard to achieve.

Embodiments of the present invention provide a source dispensing unit (SDU) that attaches to a needle assemblage, such as one used for endoscopic procedures, and that converts the assemblage to be able to be operated semi-automatically. The SDU controls the positions of three elements of the needle assemblage that are attached to the SDU: a hollow needle, a sheath that surrounds the hollow needle, and a stylet that is threaded into the hollow needle. The SDU enables independent manipulation of the sheath, the needle, and the stylet.

The independent manipulation permits control of the needle alone, for example, for initially contacting the tumor. The independent manipulation also allows controlled, relative, movement between the sheath, the needle and the stylet, so that sources may be translated to the needle distal tip, and then maintained in place, by the stylet, when the needle is retracted.

The SDU comprises a rigid bar upon which are slidingly mounted a connector to an assemblage static rod, and a connector to an assemblage handle.

The connector to the assemblage static rod enables independent adjustment of the sheath.

The connector to the assemblage handle comprises a rack and pinion assembly and the connector has a control that engages or disengages the rack and pinion. When the rack engages with the pinion, the stylet is effectively decoupled from the needle, and any translation of the pinion with respect to the rack translates the stylet independently of the needle. When the rack disengages from the pinion, the stylet is effectively coupled to the needle, so that any translation of the handle connector translates both the needle and the stylet.

The needle assemblage may be used to position multiple sources (that have been inserted into the hollow needle) into a desired location, such as a selected region of a tumor. The SDU control may then be activated to engage the rack and pinion, and while they are engaged, the handle connector may be translated proximally. As is explained below, the proximal translation of the handle connector retracts the hollow needle proximally from the sources' position, but translates the stylet distally with respect to the needle, so that a distal end of the stylet remains in place. Consequently, during the needle retraction the sources, which contact the stylet distal end, are maintained in place.

In a typical procedure, the desired implanted sources such as radioactive seeds are first loaded into the hollow needle, so as to contact the stylet in the needle. A biocompatible plug, optionally formed of a substance such as glycerin or bone wax, may be inserted into the needle and applied to the distal-most source, so as to prevent source movement from inside the needle, and so as, in the case of radioactive seeds, to reduce radiation exposure from the sources.

A physician may then insert the hollow needle into a patient so that the distal end of the needle is at a desired location in the patient, and the physician may operate the SDU to advance the stylet, thus expelling the plug and positioning the distal-most source at the needle tip, all of that without changing the needle location. The physician then retracts the hollow needle, using the SDU as described above, to position the distal-most source in the patient. By using the SDU, the physician only needs one hand to perform the operations of inserting then retracting the needle.

During a single insertion procedure, i.e., one insertion and retraction of the needle, the physician may use the SDU to insert a single source into the patient, or alternatively multiple sources consecutively, i.e., in a line. Inserting multiple consecutive sources provides radiation over a larger target region of a tumor than that of a single source.

Other actions of the SDU are described below.

### DETAILED DESCRIPTION

In the following description all directional references (e.g., upper, lower, upward, downward, left, right, top, bottom, above, below, vertical, and horizontal) are only used for identification purposes to aid the reader's understanding of the present disclosure, and do not create limitations, particularly as to the position, orientation, or use of embodiments of the invention.

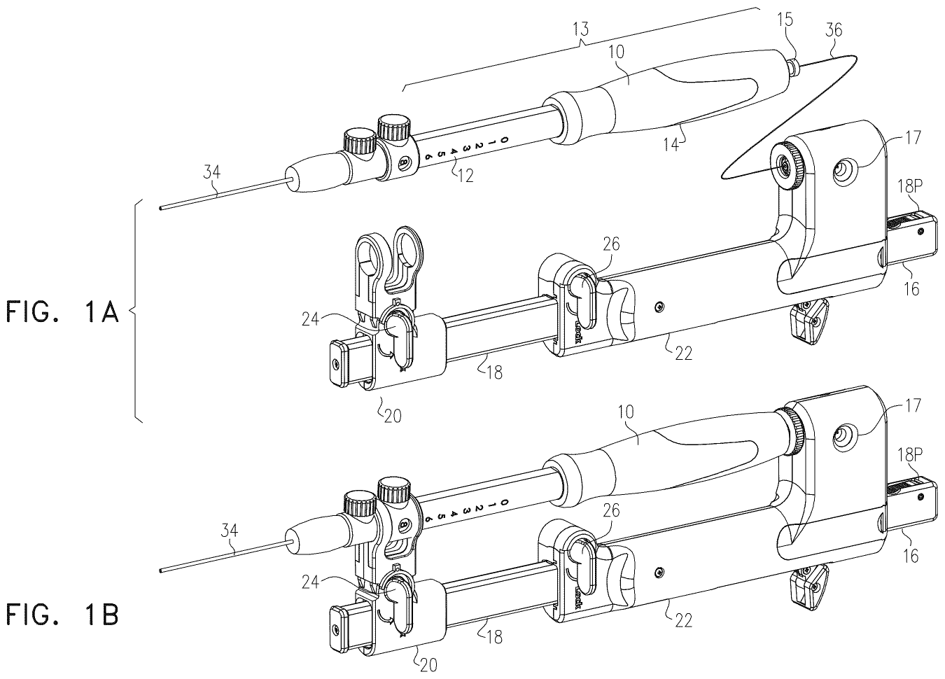

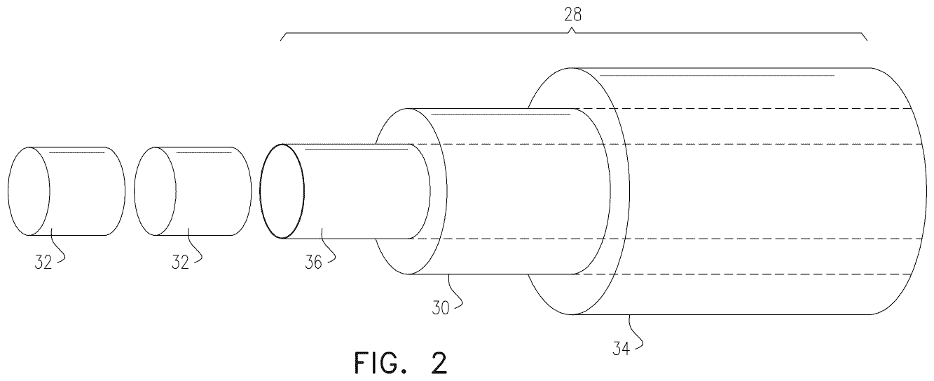

Reference is now made to FIG. 1A, FIG. 1B, and to FIG. 2. FIG. 1A is a schematic illustration of a needle assemblage 10, and of a source dispensing unit (SDU) 16 that may be attached to the needle assemblage, according to an embodiment of the present invention. FIG. 1B is a schematic illustration of SDU 16 attached to needle assemblage 10, according to an embodiment of the present invention. FIG. 2 is a schematic illustration of elements that are used by the needle assemblage, according to an embodiment of the present invention.

Needle assemblage 10 may be used for the endoscopic implantation of a plurality of sources via a single needle into a patient for therapeutic purposes such as for the placement of fiducial markers, for drug delivery, and for implantation of radiotherapy seeds into an organ of a human patient, typically into a tumor of the organ. As shown in FIG. 1A, needle assemblage 10 comprises a sheath-holding static rod 12 and a handle 14, and the handle slides around the static rod 12. Static rod 12 and handle 14 act as the basis of a source applicator, so that the rod and the handle together are also referred to herein as applicator 13. SDU 16 is configured to attach to the needle assemblage, and FIG. 1B illustrates the SDU when it is attached to the needle assemblage. As illustrated in FIG. 1A, when SDU 16 is attached to needle assemblage 10, a stylet 36, described further below, is inserted into handle 14.

SDU 16 comprises a rigid retaining bar 18, upon which two connectors are configured to slide: a static rod connector 20, which couples to static rod 12 of the needle assemblage, and a handle connector 22, which couples to handle 14. A proximal section 18P of bar 18 has a generally U-shaped cross-section, and as is described below, portions of handle connector 22 are retained within the opening of the U-shape of section 18P. Both connectors have respective locks, a first lock 24 that locks connector 20 to bar 18, and a second lock 26 that locks handle connector 22 to the bar. Handle connector 22 has an aperture 17 and the function of the aperture is described below.

In an endoscopic procedure needle assemblage 10, in addition to comprising applicator 13, comprises a set 28 of elements that are manipulated during the procedure, and details of the set are shown in FIG. 2.

As is illustrated in FIG. 2, set 28 comprises a generally cylindrical hollow needle 30, a generally cylindrical sheath 34, and generally cylindrical stylet 36. Hollow needle 30 is configured to retain a plurality of sources 32, and cylindrical sheath 34 surrounds the hollow needle. In a radiotherapy procedure sources 32 comprise radiotherapeutic seeds. Stylet 36 penetrates the hollow needle. Stylet 36, sheath 34 and needle 30 are all flexible elements, the sheath and the needle being tubular, while the stylet is a solid wire-like element. In one embodiment stylet 36 is formed from nitinol/stainless steel.

To configure needle assemblage 10, set 28 is attached to applicator 13: sheath 34 is fixedly attached to static rod 12, and hollow needle 30 is fixedly attached internally to handle 14. (Set 28 and applicator 13, when attached, form needle assemblage 10.) FIGS. 1A and 1B illustrate sheath 34 extending from the distal part of rod 12. FIG. 1A also illustrates stylet 36 entering the proximal part of handle 14; as stated above, in embodiments of the invention the stylet is inserted into the handle as source dispensing unit 16 is attached to applicator 13. The assembly of stylet 36 into handle 14 is described below.

As stated above, static rod connector 20 is coupled to static rod 12 of the needle assemblage, and once coupled, and with lock 24 unlocked, the connector may slide on bar 18. Having connector 20 unlocked, and so free to slide on bar 18, enables needle 30 move relative to sheath 34; the needle and the stylet are coupled to handle 14. When lock 24 is unlocked and lock 26 of the handle connector 22 is locked, the needle and the stylet may be moved relative to the sheath by pushing the handle connector, so sliding bar 18 in static rod connector 20.

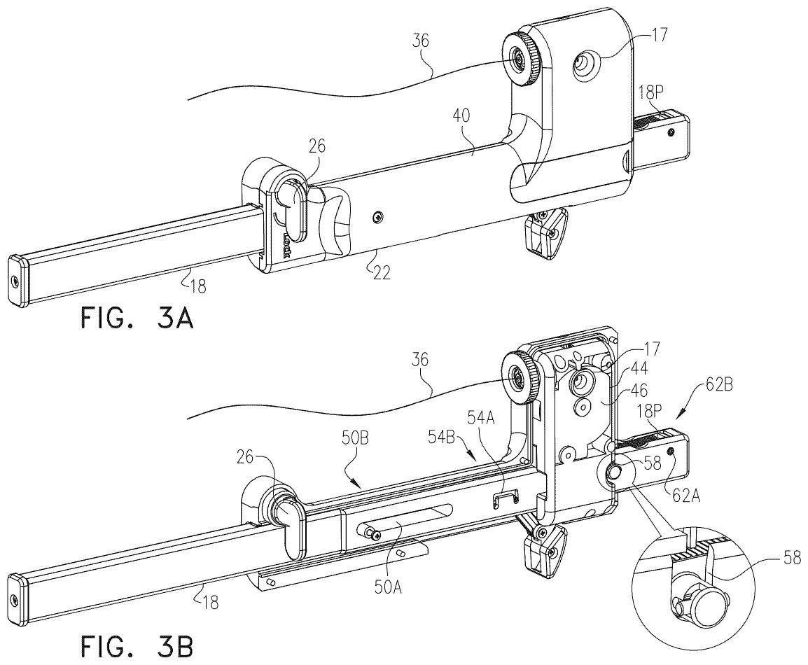

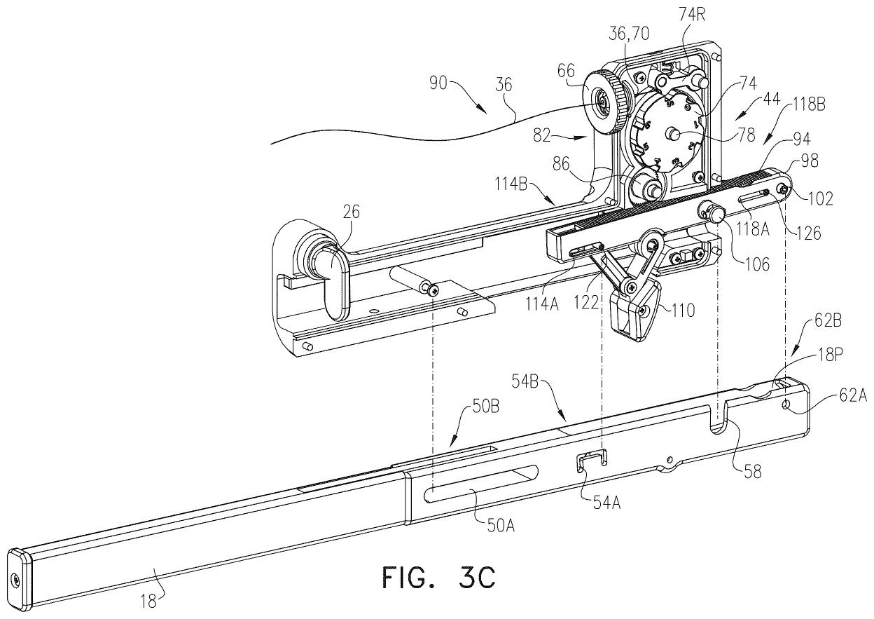

FIGS. 3A-3C are different illustrations of handle connector 22 mounted on bar 18, according to an embodiment of the present invention. FIG. 3A shows connector 22 on bar 18, and illustrates an outer container enclosure 40 of the connector. The figures also illustrate stylet 36 extending from connector 22; as is illustrated in FIG. 1A, the portion of stylet 36 extending from the connector is inserted into handle 14.

FIG. 3B has part of enclosure 40 removed, to display internal elements of the connector, as well as structure of bar 18. The internal elements of connector 22 include a gear train assembly 44, the assembly being contained in an assembly housing 46.

As is illustrated in FIG. 3B and 3C, bar 18 comprises on its front side a horizontal slot 50A, an inverted-U-shaped slot 54A, and an aperture 62A in the bar. On the back side of bar 18 there are corresponding elements i.e., a horizontal slit 50B, an inverted u-shaped slot 54B, and an aperture 62B. The functions of these elements of the bar are described below. There is also a u-shaped opening 58 in an upper part of the front side of the bar; opening 58 does not have a corresponding element on the back side of the bar.

FIG. 3C illustrates handle connector 22, with part of enclosure 40 and assembly housing 46 both being removed to show internal elements of gear train assembly 44. In addition, connector 22 and bar 18 are shown in an exploded format, where the connector is separated from the bar.

Assembly 44 comprises a luer coupling 66, which attaches to a luer coupling 15 of handle 14, shown in FIG. 1A. Prior to the procedure where apparatus 10 is to be used, stylet 36 is pre-assembled into gear assembly 44 so that a portion transfers through coupling 66 to lie in a channel 70 of the assembly, and so that a distal end of the stylet, the part illustrated in the figures as extending from coupling 66, is able to be located to a preset position in the hollow needle. The remainder of the stylet is wound round a circular ratcheted wheel 74, which has a ratchet 74R coupled to the wheel, and the end of the stylet is fixed to wheel 74. Stylet 36 is wound on wheel 74 so that rotation of the wheel, around its axis 78, in a counter-clockwise direction, moves the stylet distally, within hollow needle 30, in the direction of handle 14 and static rod 12. As is illustrated in FIG. 3C, wheel 74 has numbers on the face of the wheel, and one of these numbers is visible through aperture 17.

Coaxial with wheel 74, and behind the wheel so that it is not visible in FIG. 3C, is a gear 82 that is fixed to the wheel. Gear 82 meshes with another gear 86, which acts as a pinion of a rack and pinion assemblage 90, and which is herein also termed pinion 86. In addition to pinion 86, assemblage 90 comprises a rack 94, and the rack is held within a generally U-shaped rack support enclosure 98.

As is explained below, in an engaged state of apparatus 10 pinion 86 engages, i.e., meshes, with rack 94; in a disengaged state of the apparatus, the pinion does not mesh with the rack. When rack 94 engages with pinion 86, translation of the rack with respect to the pinion rotates the pinion, and thus wheel 74, causing stylet 36 to also translate. When the translation of the rack with respect to the pinion is a distal translation, pinion 86 rotates clockwise, so that gear 82 and its attached wheel 74 rotate counter-clockwise. The counter-clockwise rotation of the wheel translates stylet 36 distally, and it will be understood that the size of the rack and the stylet translations are equal. The distal translation of the rack with respect to the pinion may be accomplished by keeping the pinion in a fixed position, and moving the rack distally, or alternatively, by keeping the rack fixed, and moving the pinion proximally.

Rack support enclosure 98 resides in the opening of section 18P of bar 18, and a hinge pin 102, through enclosure 98 and apertures 62A and 62B of bar 18, enables the support enclosure to rotate in a vertical plane about the hinge pin. A protrusion 106 in enclosure 98 moves within u-shaped opening 58, and ensures that the enclosure remains in a vertical plane and also does not rotate more than the bound defined by the u-shaped opening. A control 110 is configured to enable an operator of apparatus 10 to rotate enclosure 98 between two states: an upper state of the enclosure, wherein rack 94 engages with pinion 86, and a lower state of the enclosure, where the rack does not engage with the pinion.

As illustrated in FIG. 3C, enclosure 98 has two horizontal slits: a slit 114A and a slit 118A, on a front surface of the enclosure, and there are two corresponding slits: a slit 114B and a slit 118B on the back of the enclosure. A first pin 122 through rack 94 connects slits 114A and 114B, and a second pin 126 through rack 94 connects slits 118A and 118B. When apparatus 10 is assembled, first pin 122 also resides in inverted U-shaped slots 54A and 54B.

Pins 122 and 126 enable rack 94 to slide horizontally, distally or proximally, within enclosure 98 within bounds defined by slits 114A and 118A (and their corresponding slits). Control 110 is connected to pin 122, and may be used to actuate the sliding of rack 94 within enclosure 98 in a ratchet-like manner, as is explained below.

An embodiment of the invention permits the following actions to be implemented on set 28 of needle elements.

### Needle Setup

Typically, to configure assemblage 10, the distal ends of sheath 34 and hollow needle 30 should be aligned. The alignment may be implemented by initially coupling static rod connector 20 to static rod 12, and by coupling handle connector 22 to handle 14, as illustrated in FIG. 1B. After the coupling, the operator of the apparatus may adjust control 110 so that rack 94 does not engage with pinion 96. The two connectors may then be slid separately on retaining bar 18, so sliding rod 12 with respect to handle 14. When the distal ends of the sheath and the hollow needle are aligned, the two connectors may be fixed in position on bar 18 using locks 24 and 26, so effectively locking sheath 34 and needle 30 together.

During the procedure needle 30 may be unlocked from sheath 34 by unlocking lock 24. Handle 14, and its attached needle 30 may then be translated distally with respect to the sheath, by pushing handle connector 22.

### Source Positioning

A plurality of sources 32 may be positioned in hollow needle 30 by siding rack 94 in a ratchet-like manner, using control 110 to slide the rack distally and proximally within rack enclosure 98. The distal and proximal movements are implemented by control 110 being used to slide pin 122 between the distal and proximal end points of slit 114A (and of slit 114B). When rack 94 is translated distally, control 110 is also used to rotate rack enclosure 98 so that rack 94 engages with pinion 86. When the rack is translated proximally, control 110 rotates rack enclosure so that rack 94 disengages from pinion 86.

Because during the rack distal translation the rack is engaged with the pinion, there is a corresponding distal translation of stylet 36, and this may be used to position sources 32 to contact the stylet. During the rack proximal translation there is no engagement, so there is no movement of stylet 36. Ratchet 74R ensures that there is no inadvertent proximal movement of the stylet during rack proximal movement.

It will be understood that this action, of pushing the stylet distally, occurs with the hollow needle fixed, so that the action enables sources 32 to be pushed to align with needle distal tip.

In a typical procedure, initially a preset number of sources may be inserted into the hollow needle, so as to contact the distal end of the stylet. The stylet distal end is positioned so that a distal end of the distal-most source is at a predetermined distance from the distal end of the hollow needle. A biocompatible plug may be inserted into the needle and applied to the distal-most source, so as to prevent source movement from inside the needle.

Once a user of apparatus 10 has inserted the distal end of the hollow needle to a desired location in a patient, the user may use control 110, as described above, (i.e., by ratcheting back and forth) to advance the stylet so as to expel the plug, and to ready apparatus for needle retraction, as described below.

### Needle Retraction

Using the actions described above, sources 32 may be positioned at the distal end of needle 30, and a user of apparatus 10 may insert the needle until the distal end of the needle is at a desired location, such as a distal part of a tumor. (Lock 24 of connector 20 should be locked on bar 18 to lock the needle location.) At this point lock 26 of connector 22 should be unlocked and control 110 may then be used to engage rack 94 and pinion 86. The user may then slide connector 22 proximally on bar 18, thus causing handle 14 and its attached needle 30 to translate proximally, i.e., the needle retracts from the distal end of the desired location. However, because of the engagement of the rack and pinion, the proximal translation of the connector causes an equivalent distal translation of stylet 36 with respect to the needle, by rotation of wheel 74.

The distal translation of stylet 36 with respect to the needle ensures, in a positive manner because the stylet distal end contacts the sources, that sources 32 remain at the desired location since the stylet distal end does not move relative to the desired location. The number on the face of wheel 74 that is visible through aperture 17 provides an indication to the user of a number of sources 32 deployed.

### Combined Needle and Stylet Translation

It will be understood that when lock 24 is unlocked and lock 26 is locked, and control 110 is not engaged, handle connector 20 may freely slide on bar 18. The sliding causes needle 30 and stylet 36 to translate, distally or proximally, together.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. An apparatus for control of a needle assemblage having a handle coupled to a hollow needle and a stylet penetrating the hollow needle, the apparatus comprising:
a retaining bar;
a connector slidingly mounted on the retaining bar, configured to fixedly attach to the handle, the connector having a control which:
in a first position causes translation of the connector proximally on the retaining bar by a preset distance to translate the handle and the hollow needle proximally by the preset distance and to translate the stylet distally by the preset distance with respect to the connector, and
in a second position causes translation of the connector on the retaining bar by the preset distance to translate the handle, the hollow needle, and the stylet by the preset distance.

2. The apparatus according to claim 1, wherein the assemblage has a static rod coupled to a sheath surrounding the hollow needle, the apparatus further comprising a further connector, slidingly mounted on the retaining bar, configured to fixedly connect to the static rod.

3. The apparatus according to claim 1, wherein the connector comprises a rack and a pinion, the pinion being coupled to a gear train retaining the stylet within the hollow needle.

4. The apparatus according to claim 3, wherein in the first position the rack and the pinion engage, and in the second position the rack and the pinion disengage.

5. The apparatus according to claim 3, comprising a rack enclosure wherein the rack is configured to slide, and wherein the control is configured to rotate the rack enclosure to a first location that places the rack in the first position and to a second location that places the rack in the second position.

6. The apparatus according to claim 5, wherein the rack enclosure rotates about a hinge fixed to the retaining bar.

7. A method for implanting at least one source into a patient, the method comprising:
providing a hollow needle having a needle distal end and a lumen configured to receive the at least one source;
inserting a stylet into the lumen;
inserting the at least one source into the lumen;
inserting the hollow needle into the patient so that the needle distal end is at a predetermined location within the patient;
feeding the stylet through the lumen to contact a proximal end of the at least one source and thereby push the at least one source so that a distal end of the at least one source aligns with the needle distal end; and
subsequent to feeding the stylet through the lumen to contact the proximal end of the at least one source, retracting the hollow needle proximally, while maintaining the stylet in position, so that the at least one source enters the patient at the predetermined location.

8. A method for controlling a needle assemblage having a handle coupled to a hollow needle and a stylet penetrating the hollow needle, the method comprising:
slidingly mounting a connector on a retaining bar;
fixedly attaching the connector to the handle; and
attaching a control, having a first position and a second position, to the connector, wherein the control:
in the first position causes translation of the connector proximally on the retaining bar by a preset distance to translate the handle and the hollow needle proximally by the preset distance and to translate the stylet distally by the preset distance with respect to the connector, and
in the second position causes translation of the connector on the retaining bar by a given distance to translate the handle, the hollow needle, and the stylet by the given distance.

9. The method according to claim 8, wherein the needle assemblage has a static rod coupled to a sheath surrounding the hollow needle, the method comprising:
slidingly mounting a further connector on the retaining bar; and,
fixedly connecting the further connector to the static rod.

10. The method according to claim 8, further comprising:
coupling a pinion of a rack and pinion being coupled to a gear train retaining the stylet within the hollow needle.

11. The method according to claim 10, wherein in the first position the rack and the pinion engage, and in the second position the rack and the pinion disengage.

12. The method according to claim 10, comprising locating the rack in a rack enclosure wherein the rack is configured to slide, and configuring the control to rotate the rack enclosure to a first location that places the rack in the first position and to a second location that places the rack in the second position.

13. The method according to claim 12, wherein the rack enclosure rotates about a hinge fixed to the retaining bar.
